(19) United States
(12) Patent Application Publication
(10) Pub. No.: US 2025/0026892 A1
(43) Pub. Date: Jan. 23, 2025

(54) NATIVE CELL MEMBRANE NANOPARTICLES

(71) Applicant: VIRGINIA COMMONWEALTH UNIVERSITY, Richmond, VA (US)

(72) Inventors: Youzhong GUO, Glen Allen, VA (US); Weihua QIU, Glen Allen, VA (US); Thi Kim Hoang TRINH, Ho Chi Minh city (VN)

(21) Appl. No.: 18/581,769

(22) Filed: Feb. 20, 2024

Related U.S. Application Data
(60) Provisional application No. 63/527,592, filed on Jul. 19, 2023.
(60) Provisional application No. 63/527,603, filed on Jul. 19, 2023.
(60) Provisional application No. 63/527,595, filed on Jul. 19, 2023.
(60) Provisional application No. 63/527,596, filed on Jul. 19, 2023.
(60) Provisional application No. 63/527,598, filed on Jul. 19, 2023.
(60) Provisional application No. 63/527,602, filed on Jul. 19, 2023.

(51) Int. Cl. C08G 81/02; G01N 33/68

(52) U.S. Cl. CPC C08G 81/024; G01N 33/6803

(57) ABSTRACT

Provided is a functionalized polymer useful for extracting, isolating, and/or purifying membrane proteins with their native protein structures being retained. Also provided are a Native Cell Membrane Nanoparticle (NCMN) system which contains the above functionalized polymer and a membrane protein and a method to characterize and/assess the native structure of the membrane protein.

### RELATED APPLICATIONS

This application claims priority from U.S. Provisional Application Ser. No. 63/527,592 filed on Jul. 19, 2023; Ser. No. 63/527,603 filed on Jul. 19, 2023; Ser. No. 63/527,595 filed on Jul. 19, 2023; Ser. No. 63/527,596 filed on Jul. 19, 2023; Ser. No. 63/527,598 filed on Jul. 19, 2023; and Ser. No. 63/527,602 filed on Jul. 19, 2023, the entire disclosure of which are incorporated herein by this reference.

### GOVERNMENT INTEREST

This invention was made with government support under grant number R01 GM132329 awarded by the National Institutes of Health (NIH). The government may have certain rights in the invention.

### FILED OF THE INVENTION

This invention relates to a Native Cell Membrane Nanoparticle (NCMN) system and methods of preparing and using such system for extracting, isolating and/or solubilizing a membrane protein. In particular, the invention provides a NCMN system which comprises one or more functionalized polymers and a method of using such polymers for extracting the membrane protein of interest while maintaining functional activity and native structure of the isolated membrane protein.

### BACKGROUND

Transmembrane proteins, including channels, transporters, receptors and enzymes, carry out a wide range of vital roles, including controlling what enters and leaves a cell and mediating intracellular communication. Consequently, they are the target of many prescribed drugs, thus there is great interest in understanding the structure and function of this class of proteins. Due to their location within a membrane bilayer and insolubility in water, extracting or solubilizing a membrane protein embedded in a lipid bilayer generally involves buffers and/or reagents containing harsh detergents or surfactants, which destabilize the membrane and interact with the protein, creating a micellar structure around the regions that would normally be in the membrane. However, the use of detergents may present several difficulties, such as regulating efficient extraction concentration of detergent without also denaturing the protein of interest, stripping away annular lipids from the protein which are crucial for function, and/or loss of lateral pressure provided by the membrane which affects both structure and function of the isolated protein. To overcome such limitations, nanodisc is used to stabilize membrane proteins after extracting from cell membrane with a detergent. Another method used in the art involves bicelle, apolipodisc or saposin, which again stabilizes the membrane protein after the detergent-based extraction. However, as apparent from the description, all of the listed strategies of stabilizing the membrane protein still involves the use of detergents in their extraction procedure. For example, U.S. Pat. No. 9,458,191 to Chromy describes a nanolipoprotein particle composition and methods to capture, solubilize and purify membrane proteins. Although the nanolipoprotein composition is able to preserve some structural integrity and activity of the isolated membrane proteins, the disclosed method in Chromy requires a step of mixing the membrane to at least three temperature transition cycles in presence of a detergent to solubilize the target protein. In addition, this method, along with other current methods for membrane protein extraction, notes an additional step of removing the detergent after the isolation of a target protein.

Recently, a styrene-maleic acid (SMA) co-polymer has been used in an alternative method that allows the membrane protein to be extracted without the use of detergents. The SMA co-polymer inserts into a biological membrane and forms small discs of bilayer encircled by the polymer, which are also called lipodisqs or native nanodiscs. The SMA co-polymer forms nanoparticles by absorbing, destabilizing and disrupting the cell membrane by a pH-dependent mechanism. The resulting SMA lipid particle (SMALP)-encapsulated proteins have been shown to be more thermostable. Despite the effective detergent-free membrane protein isolation capability of SMALP, several shortcomings of SMALP have also been apparent: i) being unstable or insoluble in certain environments (e.g., low pH, presence of divalent ions); ii) limited compatibility to only certain groups of membrane proteins (e.g., not compatible for ABC transporters); and iii) involving multiple steps in the preparation stage.

Thus, there is a need in the art for an improved transmembrane protein extraction system with simple and effective isolating properties and ability to maintain the structure and activity of the membrane protein in a detergent-free manner.

### SUMMARY OF THE INVENTION

An object of the invention is a native cell membrane nanoparticle (NCMN) system that facilitates extracting, isolating, solubilizing and/or characterizing membrane proteins, including intrinsic membrane proteins, while preserving native structures and functional activities of the proteins. The NCMN system comprises one or more functionalized polymers. Advantageous features of the present invention include excellent solubility of the NCMN system in aqueous condition and stability in various environments, especially at low temperature (e.g., 4° C.) and/or low pH (e.g., pH=4-6).

The present invention provides novel functionalized polymers for membrane protein research and technology, including effective solubilizing agents and methods.

In one aspect, the present invention provides functionalized polymers that are useful for making a NCMN system. The polymers are obtainable by:
(1) partially grafting a copolymer containing a plurality of acylating groups with an amine compound containing at least one amino group and one or more hydroxyl groups, and
(2) treating the partially grafted copolymer with a basic aqueous solution to neutralize the remaining acylating groups,

wherein the copolymer containing a plurality of acylating groups has a molecular weight of 5,000-18,000 Da and wherein the basic aqueous solution is NaOH aqueous solution, KOH aqueous solution, ammonium aqueous solution, or Na₂CO₃ aqueous solution. In certain embodiments, the amine compound used in step (1) above contains two or more hydroxyl groups. These polymers can be used for high-resolution single-particle cryo-EM structural analysis of membrane proteins in various pH conditions (even at a low pH value) and can effectively solubilize membrane proteins with the function preserved.

In another aspect, the present invention provides a method of extracting, isolating and/or solubilizing membrane proteins using one or more functionalized polymers of this invention. The method includes providing a membrane material containing at least one membrane protein and lipid associated to the membrane protein, and contacting the membrane material with one or more functionalized polymers of this invention to form a NCMN system, in which the native structure of the membrane protein is retained.

In further another aspect, the present invention provides a NCMN system, which includes (1) one or more functionalized polymers described above, and (2) a portion of a cell membrane containing at least one membrane protein and membrane lipid associated thereto; wherein the membrane protein and the associated membrane lipid are encapsulated or embedded in one or more functionalized polymers to form a nanoparticle assembly and wherein the native conformation of the membrane protein is retained in the nanoparticle assembly.

In still another aspect, the present invention provides a method of determining/characterizing the native membrane protein structure. The method includes (1) contacting a portion of a cell membrane containing at least a membrane protein and lipid associated thereto with a functionalized polymer of this invention in an aqueous solution to form a nanoparticle assembly, in which the portion of the cell membrane is encapsulated by the functionalized polymer, (2) purifying the nanoparticulate assembly from the aqueous solution, wherein the native structure of the membrane protein is retained in the isolated nanoparticulate assembly, and (3) subjecting the purified nanoparticle assembly to cryo-EM, NMR, or X-ray crystallography to determine and characterize the native structure of the membrane protein.

### BRIEF DESCRIPTION OF THE DRAWINGS

FIGS. 1A to 1C show effect of Tris-grafting degree on membrane protein solubilization and size of AcrB-NMMN particles therefrom.

FIGS. 2A to 2G show comparative effectiveness of different solubilizing agents toward BcTSPO solubilization and catalytic activity of corresponding BcTSPO therefrom in the photodegradation of PpIX.

### DETAILED DESCRIPTION OF THE INVENTION

In one aspect, this invention provides a functionalized polymer that can be used in a native cell membrane nanoparticle (NCMN) system to preserve the native structure of membrane proteins. In this system, the functionalized polymer, together with a membrane protein and lipid associated with the protein, are self-assembled into nanodiscs, where the membrane protein and lipid are wrapped, encapsulated, or encased by the functionalized polymer. The functionalized polymer of this invention is obtained by:
(1) partially grafting a copolymer containing a plurality of acylating groups with an amine compound containing at least one amino group and one or more hydroxyl groups, and
(2) treating the partially grafted copolymer with a basic aqueous solution to neutralize the remaining acylating groups,

wherein the copolymer containing a plurality of acylating groups has a molecular weight of 5,000-18,000 Da and wherein the basic aqueous solution is NaOH aqueous solution, KOH aqueous solution, ammonium aqueous solution or another basic aqueous solution. In certain embodiments, the amine compound used in step (1) above contains two or more hydroxyl groups.

The term, “polymer” or “polymer derivative”, as used herein, refers to an organic material or polymeric composition consisting of repeated one or more polymerizable units (monomers) joined together, usually in a line, like beads on a string whereas monomers are the basic building blocks of polymers. The term “nanolipoprotein particle”, “nanoparticles”, “nanodisc” or “membrane nanoparticles”, as used herein, indicates a supramolecular complex formed by membrane-forming lipid and a membrane protein and a functionalized polymer, in which the membrane protein and lipid are encased by the polymer. Both scaffold protein and target protein may constitute protein components of the NCMN system. In some embodiments, the membrane-forming lipid constitutes a lipid component of the NCMN system. Preferably, the protein and the lipid are from the same source. The term “neutralize” or “neutralization”, as used herein, indicates reaction between acid and base to form a salt. The reaction may further include hydrolysis of an acylating group. For example, an acylating group such as anhydride can be neutralized by a NaOH aqueous solution by first hydrolysis of anhydride into acid and then reaction of acid with the NaOH base to form a salt.

The functionalized polymer of this invention has a backbone derived from a copolymer which contains more than one active acylating groups that can react with a primary or secondary amino group. The term “copolymer”, as used herein, refers to a polymer derived from more than one species of monomers. It can be alternating copolymer, random copolymer, or block copolymer. Examples of the copolymer, include but are not limited to, peptide, olefin-acrylic acid copolymer or derivatives thereof, and olefin-maleic acid copolymer and derivatives thereof. The term “derivatives of a copolymer” used herein refers to copolymers wherein the active groups are replaced with their functional or chemical equivalents that lead to the same desired results. For examples, because carboxylic acid groups are equivalent to carboxylic ester and anhydride in reaction with amine to form amide or imide, derivatives of styrene-maleic acid (SMA) copolymer include styrene malate (ester) copolymer and styrene maleic anhydride copolymer. The preferred examples of copolymer are styrene-maleic acid (SMA), styrene-maleic anhydride copolymer, and diisobutylene-maleic acid (DIBMA). Diisobutylene-maleic anhydride copolymer. The term “acylating group”, as used herein, refers to a functional group containing an acyl (C═O) moiety and ready to react with, and supply the acyl group to, an organic substrate to form a new ester, amide or imide group. Examples of acylating groups include but are not limited to carboxylic acid, di- or poly-carboxylic acid, carboxylate (ester), and carboxylic anhydride.

SMA and DIBMA copolymer can be readily made from radical polymerization of styrene or dibutylene monomers and maleic acid monomers using, e.g., an organic peroxide as the initiator in a various range of molecular weight and maleic content, as evident to those skilled in the art of copolymer synthesis. Likewise, styrene maleic anhydride or diisobutylene maleic anhydride can be made from styrene or dibutylene monomers and maleic anhydride monomers. In addition, SMA, DIBMA, and their maleic anhydride derivative copolymers are also commercially available, for example, SMA® 2000 from Total Cray Valley, and Sokalan® CP 9 from BASF.

In certain embodiments, the copolymer has a number average molecular weight between 1,000 Daltons and 30,000 Daltons. In some embodiments, the copolymer has a number average molecular weight between 3,000 Daltons and 25,000 Daltons. In some embodiments, the copolymer has a number average molecular weight between 5,000 Daltons and 18,000 Daltons. In some embodiments, the copolymer has a number average molecular weight between 7,000 Daltons and 16,000 Daltons. In case of SMA or anhydride thereof, the preferred number average molecular weight is between 5,000 and 10,000, more preferably, between 6,500 and 8,000 (e.g., about 7,000 or about 7,500). In case of DIBMA and anhydride thereof, the preferred number average molecular weight is between 6,000 and 18,000, more preferably, between 8,000 and 16,000 or between 12,000 and 15,500 (e.g., about 12,000 or about 15,300).

In certain embodiments, the copolymer is an alternating copolymer. In some embodiments, the copolymer is a random copolymer. In some other embodiments, the copolymer is a block copolymer.

In certain embodiments, the copolymer features a ratio of styrene or dibutylene monomer units to maleic acid or anhydride monomer units between 10:1 to 1:10. In some embodiments, the ratio is between 5:1 to 1:5. In some embodiments, the ratio is between 4:1 to 1:1, or between 4:1 and 1.5:1, or between 2:1 and 3:1. For example, the copolymer features a ratio of styrene or dibutylene unit to maleic acid or anhydride unit being about 1:1, or about 2:1, or about 3:1 or about 4:1.

It will be understood that, due to the nature of polymerization processes, such monomer ratios are bulk averages, and are not to be taken as descriptive of a particular molecular structure having defined arrangements of monomers. Nevertheless, in general it is to be expected that the monomer types are distributed throughout the copolymer.

It will be also understood that, due to the nature of polymerization reaction, the double bonds in the monomers are transformed into single bonds in order to form additional single bonds to connect monomers. For example, a maleic acid monomer is chemically becomes a succinic acid moiety in the polymer, which is referred to herein as either “maleic acid moiety” or “succinic acid moiety” interchangeably.

In the above copolymers, all contain a plurality of acylating groups, such as monocarboxylic acid retained from an amino acid monomer, or dicarboxylic acid retained from a maleic acid monomer, or anhydride retained from a maleic anhydride monomer. They can react with an amino group or hydroxy group to form an amide, imide, or ester group.

To make the functionalized polymers of this invention, one of the above copolymers is first partially grafted with an amine compound containing at least one primary or secondary amino group and one or more hydroxyl groups. In some embodiments, the amine compound containing at least one primary or secondary amino group and two or more hydroxyl groups. It will be understood that the amino group of the amine compound reacts with an acylating group of the copolymer to form an amide (—C(O)—N) bond if it is a primary or secondary amino group, or imide (—C(O)—N—C(O)—) bond if it is a primary amino group. Examples of such amine compounds are tris[hydroxymethyl]aminomethane (Tris), 2-amino-2-methyl-1,3-propanediol (AMPDO) and 2-amino-1,3-propanediol (APDO). Some other examples include saccharide or disaccharide or polysaccharide with one hydroxy group being replaced with an amino group, such as d-glucosamine (d-GlcN), d-galactosamine (d-GalN), or d-mannosamine (d-ManN).

The phrase “partially grafted” indicates that at least one acylating group has not reacted with the amine compound and remained in the form of the original acylating group or its acid derivative. The term “grafting degree” means the percentage of the acylating groups of the copolymer that have been reacted with the amine compound. For the purpose of calculation of grafting degree, one maleic anhydride moiety contributes two acyl groups and there is considered to have two acylating groups. If one maleic anhydride moiety reacts with a primary amine to form an imide moiety or it reacts with two molecules of amine to form two amide moieties, then both acylating groups in the anhydride have been reacted. If one maleic anhydride moiety reacts with one molecule of amine to form one amide moiety and one acid moiety, then one acylating group has been reacted and the other one has not been reacted. In some embodiments, the grafting degree is 5%-90%, 5%-75%, 15-75%, 15-50%, or 25-50%. Examples of the grafting degree include about 5%, about 10%, about 15%, about 20%, about 25%, about 30%, about 35%, about 40%, about 45%, about 50%, about 55%, about 60%, about 65%, about 70%, about 75%, about 80%, about 85%, and about 90%.

The partially grafted copolymer is then treated with a basic aqueous solution to neutralize or hydrolyze/neutralize the remaining acylating group(s). Examples of the basic aqueous solution include but are not limited to NaOH, KOH, and NH₄⁺OH⁻ in water solution. It is understood by a skilled person in the art that a proper concentration and amount of basic aqueous solution is used to treat the partially grafted copolymer. After the treatment, the remaining acylating groups are transformed into ionic groups, e.g., —COO⁻Na⁺, —COO⁻K⁺, —COO⁻NH₄⁺. It will be also understood that other basic compounds, e.g., Ca(OH)₂, Mg(OH)₂, Na₂CO₃, CaCO₃, or MgCO₃ can be used in place of NaOH, KOH, or NH₄⁺OH⁻.

The functionalized polymer is partially or completely soluble in water. A stock solution of the polymer is prepared in distilled water and kept for storage at varying concentrations, e.g., 5% (w/v), or 10% (w/v) at an appropriate temperature.

In some embodiments, a functionalized polymer has been labelled to aid in identification, and the resulting NCMN system is therefore also labelled for identification. Suitable labels include, for example, fluorescent materials and radioisotopes.

Examples of the functionalized polymers are:

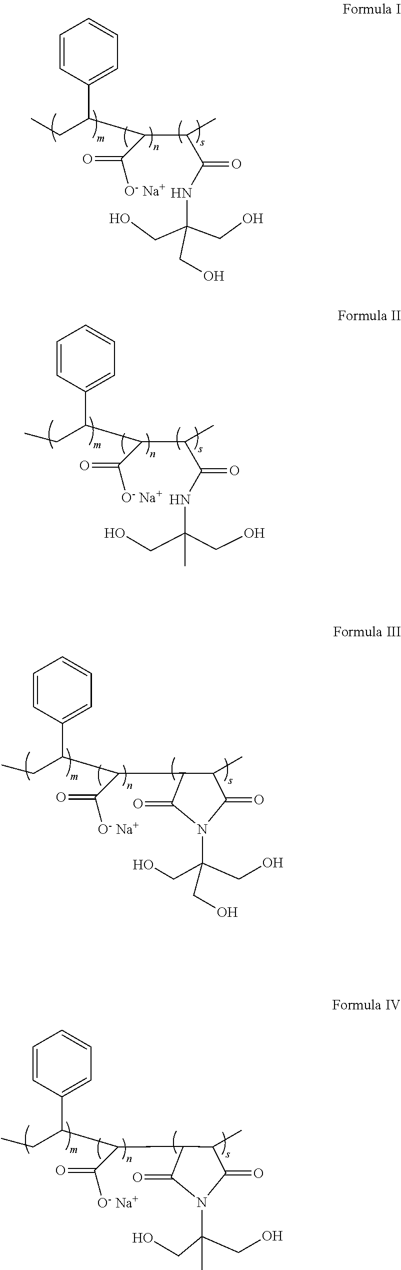

In addition to the above functionalized polymer, this invention also provides other polymers that can be used in a NCMN system to preserve the native structure of membrane proteins. Those polymers include:

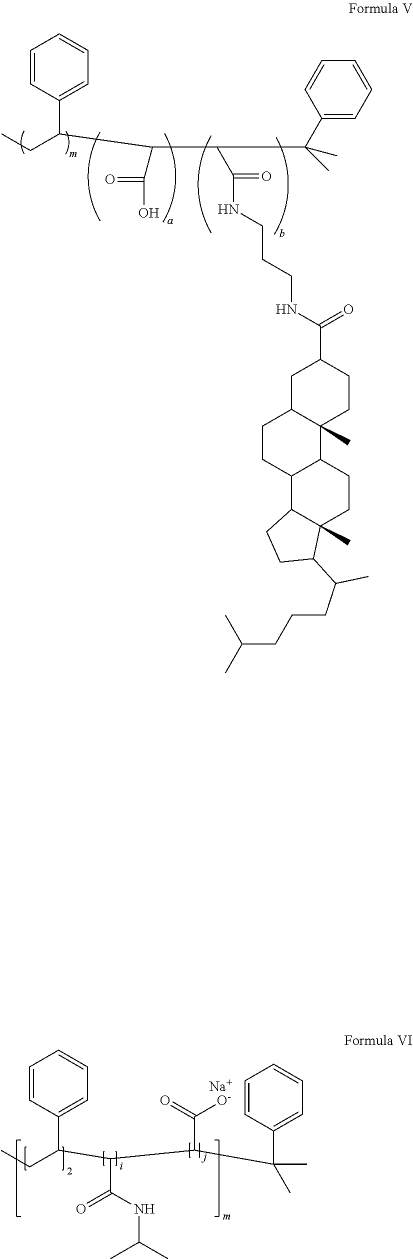

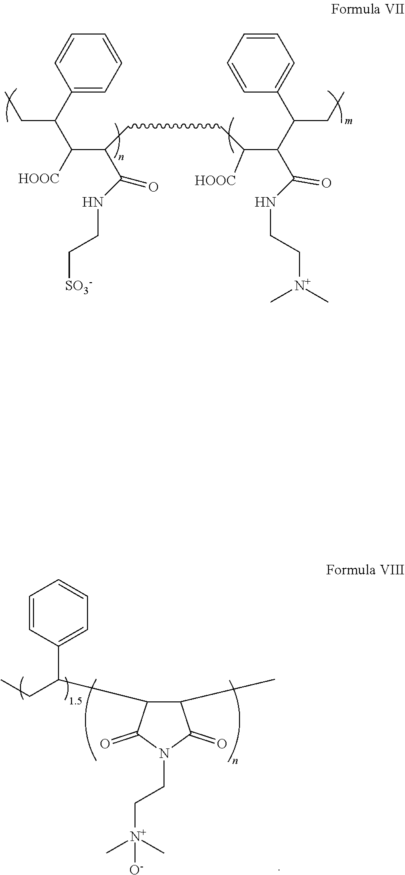

In another aspect, this invention features a NCMN system and/or a NCMN assembly, in which a membrane protein and its associated membrane lipid are encapsulated, encased, or embedded in one or more functionalized polymers described above. The terms, “encapsulated”, “encased”, and “embedded”, as used herein, refer to a state where a portion or entirety of the membrane protein of interest is enclosed, stored or hidden inside a polymer or a system comprising a polymer. The NCMN system may further comprise a suitable medium in which the NCMNs are stably dissolved or dispersed. Examples of such media include but are not limited to water, an aqueous buffer solution, and polar organic solvents like DMSO or DMF.

In preferred embodiments, the NCMN system and NCMN assembly may comprise one or more functionalized polymer-encapsulated membrane proteins in their native structures. The term “Native structure”, “Native form”, “Native environment” or “Native state”, as used herein, refers to a protein's properly folded and assembled form with operative structure and function. It is a biochemical acceptable formation of all four levels of structures including secondary, tertiary, and quaternary structures formed by weak interactions along the covalently-bonded backbone when the proteins are expressed in their designated cellular locations. The proteins in their native states with intact structures that are not altered by heat, chemicals, enzyme reaction or other denaturants are referred to “native proteins”. The “retained” native structure means that the structure of the membrane protein in the NCMN system is ≥90% identical to the structure of the protein in its native membrane environment.

The term “membrane protein, “membrane associated protein”, “target protein” or “target membrane protein” as used herein indicates any protein having a structure that is suitable for attachment to or association with a biological membrane or biomembrane (i.e., an enclosing or separating amphipathic layer that acts as a barrier within or around a cell). In particular, target proteins include proteins that contain large regions or structural domains that are hydrophobic (the regions that are embedded in or bound to the membrane); those proteins can be extremely difficult to work with in aqueous systems, since when removed from their normal lipid bilayer environment those proteins tend to aggregate and become insoluble. Accordingly, target proteins are proteins that typically can assume an active form wherein the target protein exhibits one or more functions or activities, and an inactive form wherein the target protein do not exhibit those functions/activities. Exemplary target proteins include, but are not limited to, membrane proteins, i.e., proteins that can be attached to, or associated with the membrane of a cell or an organelle, such as integral membrane proteins (i.e., proteins or assembly of proteins that are permanently attached to the biological membrane), or peripheral membrane proteins (i.e., proteins that adhere only temporarily to the biological membrane with which they are associated). Peripheral membrane proteins are proteins that attach to integral membrane proteins or penetrate the peripheral regions of the lipid bilayer with an attachment that is reversible.

Generally, integral membrane proteins may be separated from the biological membranes using detergents, nonpolar solvents, or sometimes denaturing agents. However, in preferred embodiments, the systems and methods of the present invention are substantially free of detergents or any other harsh denaturing agents. As used herein, the term “substantially free” means less than 0.01 wt. %. In other embodiments, the system is free of detergent.

The term, “membrane”, “lipid bilayer”, “membrane forming lipid” or “amphipathic lipid”, as used herein, indicates a lipid possessing both hydrophilic and hydrophobic properties that in an aqueous environment assemble in a lipid bilayer structure that consists of two opposing layers of amphipathic molecules known as polar lipids. Each polar lipid has a hydrophilic moiety (e.g., a polar group), such as a derivatized phosphate or a saccharide group, and a hydrophobic moiety (e.g., a long hydrocarbon chain). Exemplary polar lipids include phospholipids, sphingolipids, glycolipids, ether lipids, sterols and alkyl phosphocholines. Amphipathic lipids include but are not limited to membrane lipids, i.e., amphipathic lipids that are constituents of a biological membrane, such as phospholipids like dimyristoylphosphatidylcholine (DMPC) or Dioleoyl phosphoethanolamine (DOPE) or dioleoyl phosphatidylcholine (DOPC). The membrane forming lipid can assume different states in an aqueous environment, including a frozen gel state and/or a fluid liquid-crystalline state, wherein each state is associated with one or more temperatures or pHs at which the particular structural phase is detectable. The NCMN system may include at least a portion of the “native membrane” (i.e., the membrane surrounding the one or more membrane proteins of interest), which may be in a combined form with the one or more membrane proteins and at least one polymer. The “associated membrane lipid” preferably is a membrane lipid which, in a native cell, is found in the same membrane as the membrane protein. In some embodiments, the associated membrane lipid is a membrane lipid which, in a native cell, surrounds the membrane protein to form a native protein-lipid complex.

In some embodiments, the membrane protein included in the NCMN assembly or system spans across at least one half of the lipid bilayer, from one hydrophilic face to the center of the hydrophobic edge. In some cases, the membrane protein spans across the entire lipid bilayer from the first hydrophilic face to the second hydrophilic face at least once. In some cases, the membrane protein spans across the entire lipid bilayer from the first hydrophilic face to the second hydrophilic face more than once.

In yet another aspect, this invention provides a method of preparing such a NCMN system or NCMN assembly by contacting the functionalized polymer with a cellular material containing a membrane protein and its associated membrane lipid. The term “contact” refers to the act of touching, making contact, mixing, or bringing to immediate or close proximity, including at the molecular level, for example, to bring about a chemical reaction or physical change, e.g., in a solution or other reaction mixture.

The membrane protein and its associated membrane lipid in the cellular material can be from the same source or different sources. Preferably, they are from the same source. The membrane protein can be any protein that interacts with or is part of a biological lipid bilayer membrane, and can be permanently anchored or temporarily anchored to a lipid bilayer membrane.

Some exemplary membrane proteins include, but are not limited to, a G protein-coupled receptor, a 5-hydroxytryptamine receptor, an acetylcholine receptor, an adenosine receptor, an angiotensin receptor, an apelin receptor, a bile acid receptor, a bombesin receptor, a bradykinin receptor, a cannabinoid receptor, a chemerin receptor, a chemokine receptor, a cholecystokinin receptor, a Class A Orphan receptor, a dopamine receptor, an endothelin receptor, a formyl peptide receptor, a free fatty acid receptor, a galanin receptor, a ghrelin receptor, a glycoprotein hormone receptor, a gonadotrophin-releasing hormone receptor, a G protein-coupled estrogen receptor, a histamine receptor, a hydroxycarboxylic acid receptor, a kisspeptin receptor, a leukotriene receptor, a lysophospholipid receptor, a lysophospholipid SiP receptor, a melanin-concentrating hormone receptor, a melanocortin receptor, a melatonin receptor, a motilin receptor, a neuromedin U receptor, a neuropeptide FF/neuropeptide AF receptor, a neuropeptide S receptor, a neuropeptide W/neuropeptide B receptor, a neuropeptide Y receptor, a neurotensin receptor, an opioid receptor, an opsin receptor, an orexin receptor, an oxoglutarate receptor, a P2Y receptor, a platelet-activating factor receptor, a prokineticin receptor, a prolactin-releasing peptide receptor, a prostanoid receptor, a proteinase-activated receptor, a QRFP receptor, a relaxin family peptide receptor, a somatostatin receptor, a succinate receptor, a tachykinin receptor, a thyrotropin-releasing hormone receptor, a trace amine receptor, a urotensin receptor, a vasopressin receptor, U-¹⁵N Cytb5, cytochomromes such as cytochrome b5, cytochrome P450, cytochrome P450 reductase, cytochrome c, or a combination of two or more thereof.

The cellular material comprising a membrane protein and associated lipid may be whole cells. Yet, it can also be lysed or purified membrane material. Such lysed membrane material may be partly or substantially free of whole cells. Such purified membrane material may be partly, substantially or completely free of other cellular material. For example, providing purified membrane material may comprise centrifugation of lysed cells in a manner known in the art.

It has been found that the presence of DNA in a solution of macromolecular assemblies can increase viscosity and lead to difficulties in purification. Therefore, it may be advantageous to ensure that the macromolecular assemblies produced by the present method are at least substantially free of nucleic acid, either by separating the membrane material from other cellular components before macromolecular assembly, or by treatment of the liquid containing the assemblies.

An exemplary protocol of preparing Native Cell Membrane Nanoparticles (NCMN) containing membrane protein may include, but is not limited to, the following steps:
1. Resuspend membrane pellet in a suitable buffer.
2. Homogenize the resuspended cell membrane sample with a glass Dounce homogenizer
3. Add a membrane active polymer stock solution and additional buffer to bring the sample to the desired concentration.
4. Shake and ultracentrifuge the sample.
5. Collect the supernatant after ultracentrifugation is complete and load it onto a Ni-NTA column using a syringe pump.
6. Elute the protein with a suitable buffer and collect the sample.

The above-described exemplary protocol is for illustrative purposes only and is not meant to be limiting. Alternative or modified protocols and embodiments will be apparent to those of skill in the art in view of this disclosure.

In some embodiments, contacting the functionalized polymer with the cellular material is carried out at a temperature of at least 4° C., or at least 10° C., or at least 15° C. or at least 20° C. Additionally or alternatively, contacting the functionalized polymer with the cellular material may be carried out at a temperature no lower than the gel to liquid phase transition temperature of the cellular material.

In some embodiments, contacting the functionalized polymer with the cellular material is carried out at a pH of between 6.5 and 9. In some further embodiments, it is carried out at a pH of between 7 and 8.

In some embodiments, the functionalized polymer and the cellular material are independently and separately dissolved or dispersed in aqueous solutions at a suitable concentration prior to the contacting step. The aqueous solution containing the functionalized polymer is then mixed with the aqueous solution containing the cellular material. The resulting NCMN assembly is hereby solubilized or dispersed in the aqueous solution.

It is well known in biochemical research that, when working with proteins (and particularly where it is desired to maintain the proteins in their native conformations), any processing should be carried out at low temperature. Thus, processing of proteins (such as extraction) is commonly performed in a refrigerated room. The purpose of operation at low temperature is to prevent any degradation of the protein, including (but not limited to) denaturation and oxidation. However, at such low temperatures, cellular material has a tendency to form a gel-like structure, with the result that the processing is difficult to achieve and takes longer than desired.

It has surprisingly been found that the method of the invention stabilizes the protein to such an extent that processing can be carried out at higher temperatures. In particular, processing can be carried out at or above the gel to liquid phase transition temperature, thereby reducing the time required to solubilize the protein (in the form of the macromolecular assemblies) without sacrificing the protein stability.

The gel to liquid phase transition temperature is well understood by those in the art, and is known to be affected by the acyl chain lengths in the phospholipids of a phospholipid bilayer. Many lipids in bacteria and humans have chain lengths of between 12 and 14 carbon atoms, giving a transition temperature of between 2° and 40° C.

In some embodiments, mixing the polymer with the cellular material additionally comprises sonication of the mixture. This aids in mixing of the cellular material with the functionalized polymer by breaking up membranes in the cellular material.

In certain embodiments, a functionalized polymer has been labelled by e.g., fluorescent materials and radioisotopes, and, as a result, the resulting NCMN system and assembly is also labelled for identification.

In still another aspect, this invention provides a method of solubilizing one or more membrane proteins or membrane associated proteins in an aqueous medium by contacting a membrane material containing such proteins with one or more functionalized polymers in said aqueous medium to form a NCMN system in which the nanoparticles are soluble or more soluble in said aqueous medium. In further another aspect, this invention provides a method of using the NCMN system to extract, isolate, purify, and/or concentrate one or more membrane proteins for preserving the native lipid environment and/or maintaining the native structure and activity of the target membrane proteins. Each of these extracting/isolating/purifying/concentrating methods comprises steps of: contacting a cell membrane with an effective amount of one or more functionalized polymers; extracting or isolating one or more membrane proteins embedded in their native membrane within the NCMN system. For contacting a cell membrane with at least one functionalized polymer, the cell membrane and the functionalized polymer may be mixed at a ratio of about 5:1 to 1:10, preferably about 4:1 to 1:8, more preferably about 3:1 to 1:5. Such cell membranes include cell membranes in both prokaryotes and eukaryotes, and the membranes surrounding many intracellular organelles in eukaryotes.

The term “contacting” is defined above. An “effective amount” generally means an amount which provides the desired effect. In the present disclosure, an effective amount refers to a concentration or weight of the polymer to entirely or partially extract and/or purify the membrane protein of interest from the cell membrane while maintaining the protein's functional activity and native structure. For example, to extract and/or purify the membrane protein of interest, 1-100 mg, preferably 1-80 mg, or more preferably 1-50 mg of NCMN system may be used. In some embodiments, more than 100 mg of NCMN may be used. In other embodiments, less than 1 mg of NCMN may be used. In addition, an “appropriate time” refers to a time period to obtain the desired effect. For example, in the present disclosure, an appropriate time refers to a time for the functionalized polymers to extract, isolate or purify the membrane protein of interest into a NCMN system. Alternatively, an appropriate time or amount may represent the time or amount that is required for maintaining the isolated membrane protein's structure and functional activity. For example, the isolated membrane protein may be stable in a NCMN system for hours (e.g., any time between 1-24 hours), days (e.g., any day between 1-30 days), months (e.g., any month between 1-12 months) or years (e.g., 1-3 years). The phrase “one or more” is readily understood by one of skill in the art, particularly when read in context of its usage. For example, one or more of functionalized polymers refers to one to three, or one to up to four.

The terms, “extract” and “isolate”, as used herein, are used interchangeably and refer to a series of processes intended to isolate one or more proteins from a complex, such as cells, tissues, whole organisms, or cellular membranes. The process described herein refers to a method that separates one or more target membrane proteins and non-protein parts of the mixture (i.e., a portion of the membrane or a lipid bilayer). In addition, the terms may represent extraction and isolation of a specific protein of interest from all other proteins or non-protein components.

The term, “solubilize” as used herein, indicates to make a membrane protein susceptible or more susceptible to dissolve in a medium and in particular in an aqueous medium.

Accordingly, when used with reference to a membrane associated protein the term solubilize indicates making the membrane associated protein soluble or more soluble (susceptible of being dissolved) into an aqueous environment and encompasses solubilizing proteins from a pellet, a solution, a membrane fraction and any other medium and/or preparations wherein the membrane associated protein is comprised alone or in combination with other compounds and/or molecules.

The terms, “purify” and “concentrate” as used herein, indicate the process of freeing the membrane protein from other components. In particular with reference to a membrane associated protein, the term “purify” indicates the act of separating the membrane associated protein from a medium wherein the protein is comprised together with other molecules and encompasses purification of membrane associated proteins from molecular and/or biological structures such as membranes or molecular complexes. Accordingly, “purifying” a membrane associated protein into a nanoparticle indicates the act of separating the membrane associated protein from an original environment and/or cellular location into the nanoparticle particle.

The methods herein described can be used, in several embodiments, to assemble, to extract, to solubilize and/or to purify many kind of membrane proteins or membrane associated proteins of interest, including integral membrane proteins and any other proteins difficult to isolate without applying a detergent. In some embodiments, the NCMN system may comprise at least one NCMN polymer. In other embodiments, the NCMN system may comprise combinations of functionalized polymers. In some cases, the NCMN system comprises one or more functionalized polymers described above together with one or more other active compounds, for example, one or more NCMN polymers described in US 20230295349A1.

The NCMN system provides an improved tolerance to low pH conditions during the protein extraction. Further, the functionalized polymers can isolate and extract materials in a simplified step by eliminating the use of detergents. As such, detergent titration and optimization for reconstituting the isolated protein, as well as several wash steps for removal of detergent after the reconstitution, are not required, thereby increasing the efficiency and overall yield of NCMN containing liposomes.

The selection process of at least one Native Cell Membrane Nanoparticle polymer (NCMNP) in the NCMN system may be based on the pH, temperature, molecular or chemical structure, functional activity or any other factors of a specific membrane protein and/or the lipid bilayer environment of the protein. The NCMN system may be used for membrane proteins in a condition at a low pH (i.e., pH<6, 5, 4, 3), a neutral pH (i.e., pH=7) and/or a high pH (i.e., pH>8). In some embodiments, additional pH-buffer modifying solution may be included. One or more selected functionalized polymers in the system may be stable in a certain pH conditions to preserve the native structure of the membrane protein.

In some embodiments, the methods and NCMN system herein disclosed allow functionalized polymers to incorporate diverse membrane proteins, which include, but are not limited to, integral membrane proteins containing transmembrane alpha-helices and/or beta-sheet structures, as well as, peripheral and monotopic membrane proteins, G protein receptors, Type I, II and III cell-surface receptors and the likes. Membrane proteins that have single or multiple membrane spans can also be functionally solubilized into a NCMN system. Other suitable applications of the NCMN system include various biological fields, such as for detecting microorganisms and/or analysis of bacterial or viral and host protein-protein interactions. The methods of use may also include drug delivery by serving as vehicles for therapeutic based countermeasures.

In further another aspect, this invention provides a method of a method of characterizing a membrane protein or determining the structure of a membrane protein. The method includes providing a NCMN system described above, which contains a nanoparticle assembly of a functionalized polymer, a membrane protein, and lipid, and characterizing, or determine the structure of, the membrane protein in the nanoparticle assembly. This method may optionally include, prior to the characterization, isolating, concentrating or purifying the nanoparticulate assembly from the aqueous solution, wherein the native structure of the membrane protein is retained in the isolated nanoparticulate assembly.

Characterization may be either a structural characterization or a functional characterization of the membrane protein, or both. Suitable membrane protein characterization methods include solution and solid state nuclear magnetic resonance (NMR), circular dichroism (CD), electron paramagnetic resonance (EPR), Fourier transform infrared spectroscopy (FTIR), resonance Raman spectroscopy, ultraviolet-visible spectroscopy (UV/vis), cryo-electron microscopy (cryo-EM), surface plasmon Raman spectroscopy, sum frequency generation (SFG), fluorescence, including single molecule fluorescence and coherent anti-Stokes Raman (CARS), small angle x-ray scattering (SAXS), scanning electron microscopy (SEM), atomic force microscopy (AFM) and enzymatic assays membrane protein structure and dynamics can be characterized using NMR techniques. For example, membrane protein-embedded nanodiscs having a diameter of about 20 nm or less can be characterized using solution NMR and protein-embedded nanodiscs having a diameter greater than about 20 nm can be characterized using solid state NMR. Advantageously, the nanodiscs of the disclosure can include additional features for enhancing characterization by NMR, for example, the nanodisc can be characterized in that when a magnetic field is applied, the nanodisc aligns with the magnetic field and the nanodisc optionally includes a chelating group having a metal ion bound thereto which allows paramagnetic resonance characterization.

In some embodiments, characterizing comprises characterization of the native structure of a membrane protein embedded in a NCMN assembly, the characterization comprising performing at least one of solution nuclear magnetic resonance (NMR), solid state NMR, circular dichroism, electron paramagnetic resonance (EPR), Fourier transform infrared spectroscopy (FTIR), resonance Raman spectroscopy, ultraviolet-visible spectroscopy (UV/vis), cryo-electron microscopy (cryo-EM), surface plasmon Raman spectroscopy, sum frequency generation (SFG), fluorescence, small angle x-ray scattering (SAXS), scanning electron microscopy (SEM), atomic force microscopy (AFM), and an enzymatic assay. In one embodiment, the native structure is characterized by NMR. In another embodiment, the native structure is characterized by cryo-EM.

In some other embodiments, characterizing comprises characterization of the structure of the protein in a preteoliposome. The term “proteoliposome”, as used herein, refers to a system that mimics lipid membranes or liposomes to which a protein has been incorporated or inserted. The proteoliposome (PL) may represent a liposome, a vesicle or any platform that is reconstituted to preserve the embedded protein's (i.e., membrane protein) structural and functional integrities.

The polymers described herein can extract membrane proteins from cell membrane for functional study and high-resolution structure determination. The methods and systems may be used in the drug discovery field using membrane proteins as drug targets. In addition, the methods and systems described herein are Cryo-EM compatible. The methods and systems further allow to extract and purify membrane proteins in their functional forms, thus allowing reproduction and/or further analysis of membrane proteins' activity, including catalytic activity, channel transport activity, protein interaction activity, etc. As part of the NCMN system, a functionalized polymer library has been generated and other various derivatives of functionalized polymers may also be included in the system.

### EXAMPLES

Materials. Tris[hydroxymethyl]aminomethane (Tris), sodium dodecyl sulfate (SDS), ammonium persulfate (APS), 30% acrylamide/bisacrylamide solution, 37.5:1 (2.7% crosslinker), N,N,N′,N′-tetramethylethylenediamine (TEMED) and tris(2-carboxyethyl)phosphine (TCEP) were purchased from Bio-Rad. On the other hand, 4-(2-Hydroxyethyl)piperazine-1-ethanesulfonic acid (HEPES, ≥99%), glycerol (≥99.5%), nickel (II) sulfate hexahydrate (NiSO₄, >98%), imidazole (≥99%) hydrochloric acid (HCl, 36.5-38%), acetic acid (glacial, ≥99.7% w/w), methanol (MeOH, 99.8%), phenolphthalein (≥100% w/v), chloroform (≥99.8%, stabilized by ethanol) and sodium chloride (NaCl, ≥99%) with BioReagent or ACS grade were received from Fisher Chemical. DL-Dithiothreitol (DTT, ≥98% (HPLC), ≥99% (titration)), sodium hydroxide (pellets, ≥98%, reagent grade), sodium acetate (anhydrous, ≥99%, for molecular biology), protoporphyrin IX (≥95% and triethylamine (TEA, ≥99%) were obtained from Sigma-Aldrich. n-Dodecyl-β-D-maltoside (DDM, Anagrade) was received from Anatrace. Ethyl chloroformate (ClO₂Et, 99%), deuterium oxide (D₂O, 99.9 atom % D), and N, N-dimethylformamide (DMF, anhydrous, 99.8%) were ordered from Acros that were stored in a dark brown glass bottle with a self-sealing septum. Poly(styrene-co-maleic anhydride) with a 2:1 ratio of styrene to MAnh (SMAnh, acid number: 355 mg KOH·g⁻¹) were sold by Cray Valley. Before preparing different buffer solutions, water was double-deionized by a Millipore Milli-Q system to produce 18 MΩ deionized water (DI H₂O).

### Example 1: Synthesis of NCMN Polymers

NCMNP2a-5, NCMNP2a-25, and NCMNP2a-50: Tris with a given amount (0.19 g, 0.96 g, and 1.92 g for NCMNP2a-5, NCMNP2a-25, and NCMNP2a-50, respectively) was well dissolved in 75 mL DI H₂O. Then, it was singly charged into a 250 mL flask containing SMAnh (5 g, 15.84 mmol of MAnh, 1 equiv.). Afterward, the mixture was heated under reflux at 110° C. for 4 h. NCMNP2a-50 clarified at this state while NCMNP2a-5 and NCMNP2a-25 were still turbid, indicating that a high amount of MAnh had remained unchanged. Therefore, these were further reacted with NaOH 1 M (25 mL) at 110° C. for 2 h until getting a transparent solution. All modified copolymers were cooled to room temperature and recovered by precipitating into HCl 12 N (pH<2), before being washed 3 times in DI H₂O. The precipitates were next re-dissolved in NaOH 0.6 M and precipitated in HCl again. In the final step, the pH of each solution was adjusted to 7.8-8 and filtered through a 0.2 μm MilliporeSigma™ filter paper before lyophilization that afforded white powders in high yield (>97%).
NCMNP2a-70: There are two subsequent steps in the preparation of NCMNP2a-70. One is a simple ring-opening reaction of SMAnh using Tris as a nucleophile agent, and the other is an amide coupling reaction between carboxylic acid and amine. In the first step, to a round-bottom flask containing SMAnh (5 g, 15.84 mmol of MAnh, 1 equiv.) in 50 mL anhydrous DMF, Tris (2.015 g, 16.63 mmol, 1.05 equiv.) in 25 mL hot DMF was added and agitated at 110° C. for 4 h until obtaining a clear solution. In the second step, the yellow solution was cooled to 0° C. in an ice bath for 30 min, degassed, and placed under nitrogen before TEA (3.31 mL, 23.76 mmol, 1.5 equiv.) was added. The solution was agitated for 1 h following a dropwise addition of C1CO₂Et (2.26 mL, 23.76 mmol, 1.5 equiv.). After stirring for a further 1 h, Tris (0.84 g, 6.97 mmol, 0.44 equiv.) dissolved in 25 mL hot DMF was quickly added, and then the reaction mixture was continued agitating for 24 h at room temperature. Afterward, the polymer was purified through precipitation in 150 mL of cold acetone (twice). The white solid was collected and re-dissolved in 50 mL DI H₂O. In the final step, the pH of the solution was adjusted to 7.8-8 and filtered through a 0.2 μm MilliporeSigma™ filter paper before lyophilization that afforded white powder in high yield (>90%).

### Determination of Tris Grafting Degree

The calculation for Tris grafting degree. The percentage of Tris incorporated into the SMA backbone was calculated following the formula below:

$$Tris grafting percentage = \frac{{y}_{n}}{{y}_{n} + {z}_{n}} \times 100 \qquad ( 1 )$$

where: yₙ and zₙ (n=1, 2, 3 or 4) are repeating units calculated based on integrating chemical shifts in ¹H-NMR spectra. Due to proton exchange with deuterium oxide (D₂O) solvent, the NH and OH protons from amide and free hydroxyl groups, respectively, can be entirely ignored. During hydrophilic modification, the number of aromatic protons of NCMNP2a-x is consistent with its from SMA. Therefore, the xₙ value (xₙ=18.6) and the relationship between yₙ and z₁ (yₙ+zₙ=16.2) are determined based on the characterization of commercial SMAnh and its hydrolysis SMA by ¹H-NMR (see the determination of repeating units section). Therefore, yₙ, zₙ, and Tris grafting percentages for each synthesized polymer were determined in detail, shown below and summarized in Table 1.

### Repeating Units:

$${x}_{n} = 18.6 \qquad ( 2 )$$

$${y}_{n} + {z}_{n} = 16.2 \qquad ( 3 )$$

### Total Integration of Aliphatic Protons:

$$3 {I}_{H b} + ( 2 {I}_{H c} + {I}_{H d} ) \times {x}_{n} + ( {I}_{H e} + 6 {I}_{H f} ) \times {y}_{n} + {I}_{H g} \times {z}_{n} + 6 {I}_{H i} = \sum Aiphatic protons 3 {x}_{n} + 7 {y}_{n} + {z}_{n} = \sum Aiphatic protons - 9 \qquad ( 4 )$$

From (1)-(4) above, we calculated the Tris degrees of NCMNP2a-x and data are summarized in Table 1.

TABLE 1
Calculation of sulfonating degree of NCMNP2a-x and summary for QSAR analysis of
NCMNP2a-x polymers toward their stability and performance in AcrB solubilization.

| | Amine/ | | | | Tris- | | | |
|---|---|---|---|---|---|---|---|---|
| | MAnh | | | | grafting | | Tolerance | NCMN |
| | feeding | | | | degree | Optimum | to [Ca²⁺] | particle |
| Polymer | ratio^{a} | Σₚᵣₒₜₒₙₛ^{Aiphatic} | yₙ | zₙ | (%) ^{b} | pH^{c} | (mM) ^{c} | morphology^{d} |
| NCMNP2a-5 | 0.05 | 86.67 | 0.95 | 15.25 | 5.9 | >4 | <1.5 | Single |
| | | | | | | | | particle |
| NCMNP2a-25 | 0.25 | 104.77 | 3.96 | 12.24 | 24.4 | >3 | <1.5 | Single |
| | | | | | | | | particle |
| NCMNP2a-50 | 0.5 | 128.43 | 7.90 | 8.30 | 48.8 | >2 | <2 | Single |
| | | | | | | | | particle |
| NCMNP2a-70 | 0.7 | 144.82 | 10.64 | 5.56 | 65.7 | >1 | <5 | Large |
| | | | | | | | | patch |

^{a}Molar ratio.
^{b} Experimental grafting percentage of amine calculated based on ¹H-NMR characterization.
^{c} Stability of NCMNP2a-x polymers in various buffer environments determined based on turbidity experiments.
^{d}Morphology of AcrB-NCMN particles visualized using negative-TEM analysis.

Similarly, 2-amino-2-methyl-1,3-propanediol (AMPDO) and 2-amino-1,3-propanediol (APDO) were respectively grafted onto hydrophilic residues of SMA with a grafting degree of 50%. Membrane solubilization testing revealed that both polymers were effective solubilizers toward different membrane proteins. Furthermore, they both showed notable improvement in pH sensitivity (stable at pH>3).

In addition, polymers of Formulas V-VIII were prepared in similar manners. More specifically, the polymer of Formula V was prepared via facile grafting of cholesterol-amine onto hydrophilic residues of SMA with a grafting degree of 5.4%; the polymer of Formula VI with a grafting degree of 47.4% was prepared by reacting SMA with isopropylamine (IPA) in the solvent of CHCl₃ at 10° C. for 4 hrs; the polymer of Formula VII was prepared by first reacting SMA with N,N-dimethylethylenediamine (DMEDA) at 100° C., then hydrolyzed with NaOH at 110° C., and finally reacting with Taurine in the presence of EDC at room temperature; and the polymer of Formula VIII was prepared by reacting SMA with DMEDA followed by the oxidation with H₂O₂ at 60° C. Membrane solubilization testing revealed that all these four polymers were effective solubilizers toward different membrane proteins. Furthermore, the polymer of Formula VI showed a notable improvement in pH sensitivity (stable at pH>3) and the polymer of Formula VIII showed notable improvement in pH- and divalent cation-sensitivity.

### Example 2: Expression, Solubilization and Purification of AcrB Protein from the Native Cell Membrane and AcrB-NCMN Particles Characterizations

The expression and cell lysis of AcrB and Bacillus cereus TSPO (BcTSPO) membrane were carried out based on the protocol described in Y. Guo, et al., Science, 2015, 347, 551-555 and K. Kroeck, et al., Journal of Visualized Experiments. 2020, DOI: 10.3791/61298, e61298. Following a typical solubilization procedure, 1 g membrane fraction was suspended in 10 mL NCMN Buffer A and then homogenized by using a Dounce homogenizer. Consequently, the suspended membrane was transferred to a 50 mL polypropylene tube and mixed with NCMNP2a-x for a final concentration of 2.5% w/v. After shaking the sample for 2 h at 20° C., the insoluble species were centrifuged at 64,000×g (for NCMNP2a-70 media) and 200,000×g (for all other media) for 1 h 20° C. The collected supernatant was loaded onto a 5 mL Ni-NTA column (GE Healthcare) pre-equilibrated with NCMN Buffer A at a flow rate of 0.5 ml·ml⁻¹. Then, the column was washed with 30 mL of NCMN Buffer B and 30 mL of NCMN Buffer C before the protein was eluted with a mixture buffer of the NCMN Buffer C and NCMN Buffer D (1:1 v/v).

All the buffers were filtered with 0.22 μm MCE Membrane (MF-Millipore™) before use and their compositions are listed below:
NCMN Buffer A: 50 mM HEPES, pH 8.4, 500 mM NaCl, 5% glycerol, 20 mM imidazole, 0.1 mM TCEP
NCMN Buffer B: 25 mM HEPES, pH 7.8, 500 mM NaCl, 40 mM imidazole, 0.1 mM TCEP
NCMN Buffer C: 25 mM HEPES, pH 7.8, 500 mM NaCl, 75 mM imidazole, 0.1 mM TCEP
NCMN Buffer D: 25 mM HEPES, pH 7.8, 500 mM NaCl, 500 mM imidazole, 0.1 mM TCEP

### AcrB-NCMN Particles Characterizations

Transmission electron microscopy (TEM): Carbon-coated copper grids (400 nm mesh) were glow-charged for 30 s before separately loading 3.5 μL of AcrB-NCMN particles with 0.1 mg-mL⁻¹ of protein concentration and left for 1 min to absorb fully. Then, the grid surface was rinsed 3 times with DI H₂O followed by staining twice with filtered 2% w/v uranyl acetate for 1 min. Filter papers were utilized to remove surplus liquid in each stage that was continued air-dry for at least 1 min. Images were taken by a Tecnai F20 UVA transmission electron microscope working at 120 kV. Each sample was imaged at 62,000× magnification on a 4k×4k CCD camera.
Lipid extraction: Lipids from AcrB-NCMN particles were directly extracted by chloroform/MeOH at 4° C., as previously described with some modification. In detail, to 0.5 mL of NCMN particles ([AcrB]=1.5 mg·mL⁻¹, 1 volume), 2 volumes of chloroform and 1 volume of MeOH were added and stirred for 1 h before adding one more volume of chloroform. Then, the solution was continuously stirred for a further 15 min followed by the addition of 1 volume of DI H₂O. After centrifuging at 13,000×g for 10 min, the organic layer was rinsed three times with 2 volumes of cold DI H₂O. Afterward, the organic phase was characterized by electrospray ionization mass spectrometry (ESI-MS). Another lipid extraction was performed with a native cell membrane using a similar method to compare lipid composition.
Electrospray ionization mass spectrometry (ESI-MS). The lipids were separated by reverse-phase LC using a Thermo Scientific Accucore Vanquish C18+2.1 (i.d.)×150 mm column with 1.5 μm particles. The UHPLC used a binary solvent system at a 0.26 mL/min flow rate with a column oven set to 55° C. Before injection of the sample, the column was equilibrated for 2 min with a solvent mixture of 99% mobile phase A1 (CH₃CN/H₂O, 50/50, v/v, with 5 mM ammonium formate and 0.1% formic acid) and 1% mobile phase B1 (CH₃CHOHCH₃/CH₃CN/H₂O, 88/10/2, v/v/v, with 5 mM ammonium formate and 0.1% formic acid) and after the sample injection (typically 10 μL). The A1/B1 ratio was maintained at 99/1 for 1.0 min followed by a linear gradient to 35% B1 over 2.0 min, then a linear gradient to 60% B1 over 6 min followed by a linear gradient to 100% B1 over 11 min. At this step the run was held at 100% B1 for 5 min, followed by a 2.0 min gradient return to 99/1 (A1/B1). The column was re-equilibrated with 99/1 (A1/B1) for 2.0 min before the next run. Each sample was injected twice for analysis in both positive and negative modes. For the initial full scan MS (range 300 to 200 m/z), the resolution was set to 120,000 with a data-dependent MS2 triggered for any analyte reaching 3e⁶ or above signal. Data-dependent MS2 was collected at 30,000 resolutions. Data were analyzed using Thermo Scientific's Lipid Search 4.2 software.
Cryo-EM grid preparation, data collection and model building. A 2 μL of purified AcrB (1 mg·ml⁻¹) was added onto a pre-glow discharged Ultrafoil 0.6/1.0 grids, blotted for 3.5-4 s, and vitrified in liquid ethane by using FEI Vitrobot IV. Data were acquired using an FEI Titan Halo 80-300 transmission electron microscope at 300 kV equipped with a Gatan K3 Summit direction electron detector camera at New York Structural Biology Center. Images of AcrB in NCMNP2a-5 (pH=7.8), NCMNP2a-50 (pH=7.8), and NCMNP2a-50 (pH=5) were gained in electron counting mode with a pixel size of 0.87, 0.88 and 0.846 Å, respectively. Particles were picked using WARP 1.0.9. The WARP written as goodparticles.star files were imported to cryoSPARC v.3.1. The 2D classification was done to separate good particles from false positives. A de novo ab initio model for each sample was then generated. Heterogeneous refinement using AcrB and decoy volumes was done to separate good particles from false positives further. The final particles and corresponding volume were subjected to Non-uniform refinement resulting in the final reconstruction of AcrB at 3.51, 3.05, and 3.07 Å for in NCMNP2a-5 (pH=7.8), NCMNP2a-50 (pH=7.8) and NCMNP2a-50 (pH=5), respectively. In the next step, Chimera was utilized to fit the initial maps with our previous AcrB structure in SMA2000 (PDB 6BAJ). Before manually fitting in Coot, these maps were sharpened within Phenix. The atom models were built accordingly and refined several rounds with Phenix before deposits.

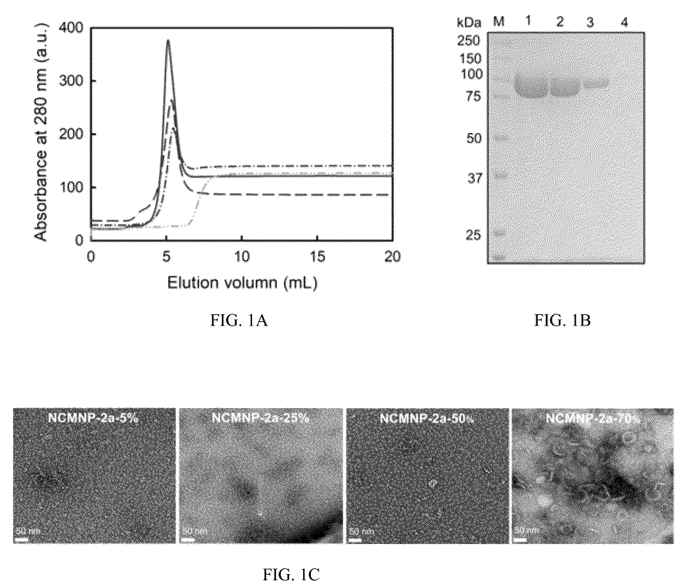

FIGS. 1A to 1C show effects of polymer grafting degree on membrane protein solubilization and size of AcrB-NCMN particles therefrom. FIG. 1A shows Ni-NTA affinity chromatography profiles of purified AcrB in different NCMN polymers: NCMNP-2a-5%

( [symbol] ), NCMNP-2a-25% ( [symbol] ), NCMNP-2a-50% ( [symbol] ), and NCMNP-2a-70%

( [symbol] ). FIG. 1B shows analysis of AcrB-NCMN particles on SDS-PAGE along [symbol] with protein marker (M). Lanes 1, 2, 3, and 4 correspond to purified AcrB extracted by NCMNP2a-5, NCMNP2a-25, NCMNP2a-50, and NCMNP2a-70, respectively. FIG. 1C shows negative stain analysis of AcrB particles extracted by NCMNP2a-x polymers (x=5, 25, 50, and 70). The bottom-left scale bar represents 50 nm.

In conclusion, it has been demonstrated polymers is suitable for high-resolution structure determination of the model membrane protein, AcrB, at different pH conditions (within a broad pH range). Variation of the grafting ratio of Tris on the SMA2000 backbone determines the pH sensitivity of the NCMNP2a-x polymers and also governs the cell membrane solubilization efficiency and subsequent size of NCMN particles. When solubilizing the cell membrane using NCMNP2a-70, very few AcrB-NCMN particles were obtained, and the size of such particles was much larger than that obtained using NCMNP2a-5 and NCMNP2a-50. Non-targeted membrane proteins were also encased as contaminants in such lipid bilayer patches. However, NCMNP2a-70 may be particularly useful when studying large membrane protein complexes. For example, these large and enriched cell membrane patches may be suitable for cryo-EM tomography analysis.

In contrast, the low Tris-grafted NCMNP2a-x polymers (≤50% of Tris) favored the solubilization of AcrB and its endogenous lipids into single particles suitable for single-particle cryo-EM analysis and can produce atomic-level membrane protein structures. Notably, Iα helix™ residues at the outer of helical bundles were fully resolved using NCMNP2a-x. In contrast, one of the Iα helix™ within the three subunits gave inferior EM density when using SMA2000. The lipid bilayer plugs were well preserved in all three cryo-EM structures at different pH conditions.

### Example 3: Expression, Solubilization and Purification and Activity Assay of BcTSPO

The expression, solubilization, and purification of Bacillus cereus TSPO (BcTSPO) membrane were carried out in a manner similar to the protocol in Example 2.

Before performing the activity assays, different reaction solutions were prepared following the procedures below:
PpIX stock solution was prepared as follows: adding 1 μL of saturated PpIX in DMSO to 199 μL of Buffer E (pH 7.8) and then taking 50 μL of this mixture and diluting 4× with Buffer E (pH 7.8)
Buffer E contains 40 mM HEPES, 100 mM NaCl, 0.1 mM TCEP, and 0.05% w/v DDM. The pH value was adjusted to 7.8, 5, 4, or 3, depending on the target pH of the experiments. The solution was filtered with 0.22 μm MCE membrane before use.

The enzyme activity tests were conducted following the protocol described in Y. Guo, et al., Science, 2015, 347, 551-555 with modifications. Typically, 100 μL of BcTSPO ([BcTSPO]1 mg·ml⁻¹) was mixed with 129 μL of Buffer E (pH 7.8, pH 5, pH 4 or pH 3) and 1 μL of PpIX stock solution. This media was transferred into a 5 mm square quartz cuvette and then incubated in the dark for 5 min. The reaction was initiated by opening the shutters and exciting the mixture at 405 nm. The fluorescence emission was monitored from 550-750 nm.

All experiments were conducted on a Shimadzu RF-53301PC fluorescence spectrometer. The samples were illuminated at 405 nm with excitation bandwidth at 1.5 nm. On the other hand, the emission spectra were recorded with emission bandwidth at 3 nm.

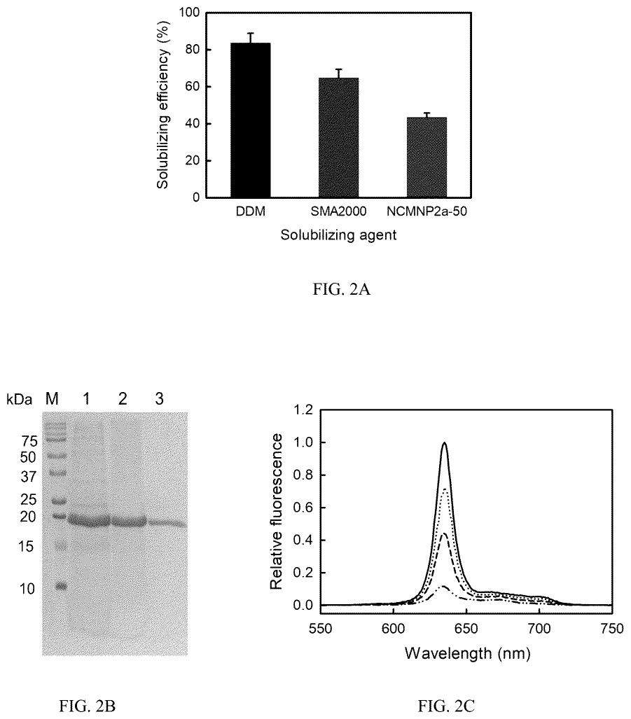

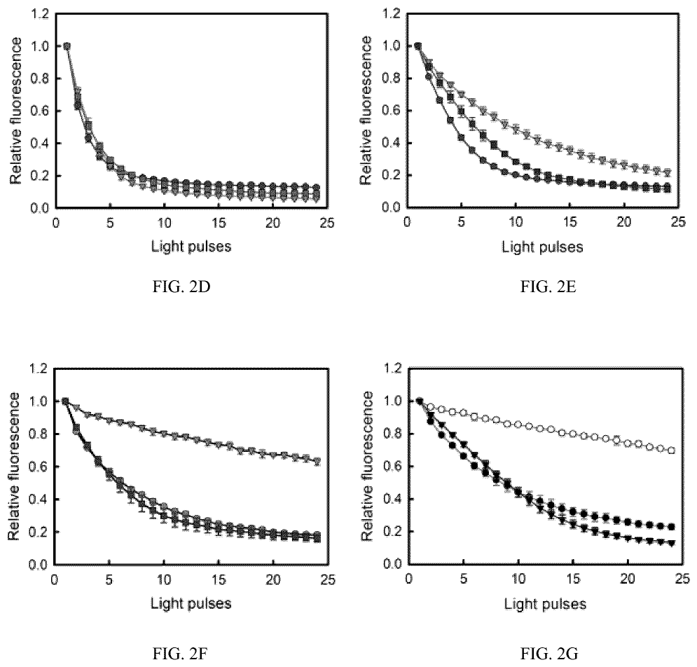

FIGS. 2A to 2G show comparative effectiveness of different solubilizing agents toward BcTSPO solubilization and catalytic activity of corresponding BcTSPO therefrom in the photodegradation of PpIX. FIG. 2A illustrates solubilization effectiveness of recombinant protein BcTPSO in DDM, SMA2000 and NCMNP2a-50. FIG. 2B is visualization of purified BcTSPO particles on Coomassie-stained SDS-PAGE compared to protein ladder (M). Lane 1, 2 and 3 correspond to BcTSPO particles extracted by DDM, SMA2000 and NCMNP2a-50, respectively. FIG. 2C provides representative fluorescent emission spectra of PpIX associated with BcTSPO-NCMN particles upon excitation at 410 nm with 1 pulse ( [symbol] ), 4 pulses ( [symbol] ), 8 pulses ( [symbol] ), and 24 pulses ( [symbol] ). FIGS. 2D to 2G illustrate photodegradation of PpIX in the presence of BcTSPO at various pH conditions. Decays of PpIX at 632 nm as a function of the light pulse at pH=7.8 in DDM ( [symbol] ), SMA2000 ( [symbol] ), and NCMNP2a-50 ( [symbol] ) are shown in FIG. 2D. Decays of PpIX at 632 nm as a function of the light pulse at pH=5 in DDM ( [symbol] ), SMA2000 ( [symbol] ) and NCMNP2a-50 ( [symbol] ) are shown in FIG. 2E. Decays of PpIX at 632 nm as a function of the light pulse at pH=4 in DDM ( [symbol] ), SMA2000 ( [symbol] ) and NCMNP2a-50 ( [symbol] ) are shown in FIG. 2F. Decays of PpIX at 632 nm as a function of the light pulse at pH=3 in DDM ( [symbol] ), SMA2000 ( [symbol] ) and NCMNP2a-50 ( [symbol] ) are shown in FIG. 2G.

In conclusion, it has been demonstrated that NCMNP2a-x could be used to analyze the enzyme activity of BcTSPO at different pH conditions.

It is to be understood that this invention is not limited to any particular embodiment described herein and may vary. It is also to be understood that the terminology used herein is for the purpose of describing particular embodiments only, and is not intended to be limiting, since the scope of the present invention will be limited only by the appended claims.

Where a range of values is provided, it is understood that each intervening value between the upper and lower limit of that range (to a tenth of the unit of the lower limit) is included in the range and encompassed within the invention, unless the context or description clearly dictates otherwise. In addition, smaller ranges between any two values in the range are encompassed, unless the context or description clearly indicates otherwise.

Unless defined otherwise, all technical and scientific terms used herein have the same meaning as commonly understood by one of ordinary skill in the art to which this invention belongs. Representative illustrative methods and materials are herein described; methods and materials similar or equivalent to those described herein can also be used in the practice or testing of the present invention.

All publications and patents cited in this specification are herein incorporated by reference as if each individual publication or patent were specifically and individually indicated to be incorporated by reference, and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual dates of public availability and may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as support for the recitation in the claims of such exclusive terminology as “solely,” “only” and the like in connection with the recitation of claim elements, or use of a “negative” limitations, such as “wherein [a particular feature or element] is absent”, or “except for [a particular feature or element]”, or “wherein [a particular feature or element] is not present (included, etc.) . . . ”.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

What is claimed is:

1. A functionalized polymer obtained by
(1) partially grafting a copolymer containing a plurality of acylating groups with an amine compound containing at least one primary or secondary amino group and two or more hydroxyl groups, and
(2) treating the partially grafted copolymer with a basic aqueous solution to neutralize the remaining acylating groups,
wherein the copolymer containing a plurality of acylating groups has a molecular weight of 5,000-18,000 Da and wherein the basic aqueous solution is NaOH aqueous solution, KOH aqueous solution, or ammonium aqueous solution.

2. The functionalized polymer of claim 1, wherein the amine compound is selected from the group consisting of tris(hydroxymethyl) aminomethane or 2-amino-2-methyl-1,3-propyanediol.

3. The functionalized polymer of claim 1, wherein the copolymer containing a plurality of acylating groups is selected from the group consisting of styrene-maleic acid copolymer, styrene-maleic anhydride, di-isobutylene-maleic acid, and di-isobutylene-maleic anhydride.

4. The functionalized polymer of claim 1, wherein the backbone is a copolymer radical having a number average molecular weight of 7,000-16,000 Da.

5. The functionalized polymer of claim 1, wherein the grafting degree of step (1) is from 5% to 75%.

6. The functionalized polymer of claim 1, wherein the amine compound is grafted onto the copolymer by forming amide or imide bonding.

7. The functionalized polymer of claim 6, wherein the grafting degree of step (1) is from 5% to 75%.

8. The functionalized polymer of claim 7, wherein the basic aqueous solution used in Step (2) is NaOH aqueous solution.

9. The functionalized polymer of claim 1, wherein the grafting degree of step (1) is from 15% to 75%.

10. The functionalized polymer of claim 1, wherein the grafting degree of step (1) is from 15% to 50%.

11. The functionalized polymer of claim 1, wherein the grafting degree of step (1) is from 25% to 50%.

12. The functionalized polymer of claim 1, wherein the functional copolymer has a structure selected from a group consisting of:

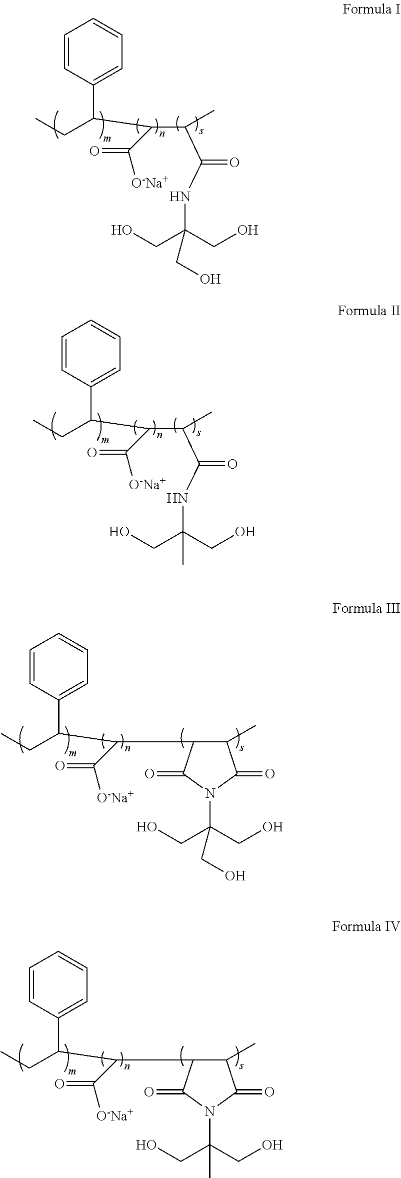

13. The functionalized polymer of claim 3, wherein the ratio of styrene monomer units and maleic monomer units of the copolymer backbone is between 0.5:1 and 10:1.

14. The he functionalized polymer of claim 13, wherein the grafting degree of step (1) is from 5% to 75%.

15. The functionalized polymer of claim 14, wherein the amine compound having two or more hydroxyl groups of step (2) is grafted onto the copolymer by forming amide or imide bonding.

16. The functionalized polymer of claim 1, wherein the amine compound is selected from the group consisting of tris(hydroxymethyl) aminomethane or 2-amino-2-methyl-1,3-propyanediol.

17. The functionalized polymer of claim 7, wherein the basic aqueous solution used in Step (2) is NaOH aqueous solution.

18. The functionalized polymer of claim 1, wherein the grafting degree of step (1) is from 15% to 75%.

19. The functionalized polymer of claim 1, wherein the grafting degree of step (1) is from 15% to 50%.

20. The functionalized polymer of claim 1, wherein the grafting degree of step (1) is from 25% to 50%.

21. A native cell membrane nanoparticle system comprising:
(1) one or more functionalized polymers of claim 1, and
(2) a portion of a cell membrane containing at least one membrane protein and membrane lipid associated thereto;
wherein the one or more functionalized polymers encapsulate the membrane protein and the associated membrane lipid to form a nanoparticle assembly and wherein the native structure of the membrane protein is retained in the nanoparticle assembly.

22. The native cell membrane nanoparticle system of claim 21, wherein the nanoparticle assembly is soluble in water.

23. The native cell membrane nanoparticle system of claim 21, wherein the cell membrane protein is selected from a group consisting of integral membrane proteins, peripheral and monotopic membrane proteins, G protein receptors, and Type I, II and III cell-surface receptors.

24. The native cell membrane nanoparticle system of claim 18, wherein the system is substantially free of a detergent.

25. A method of extracting a membrane protein comprising:
(1) contacting a portion of a cell membrane containing at least one membrane protein and lipid associated thereto with a functionalized copolymer of claim 1 in an aqueous solution to form a nanoparticle assembly, in which the portion of the cell membrane is encapsulated by the functionalized copolymer, and
(2) isolating the nanoparticulate assembly from the aqueous solution;
wherein the native structure of the membrane protein is retained in the isolated nanoparticulate assembly.

26. A method of characterizing the native structure of a membrane protein, comprising:
(1) contacting a portion of a cell membrane containing at least a membrane protein and lipid associated thereto with a functionalized copolymer of claim 1 in an aqueous solution to form a nanoparticle assembly, in which the portion of the cell membrane is encapsulated by the functionalized copolymer,
(2) purifying the nanoparticulate assembly from the aqueous solution, wherein the native structure of the membrane protein is retained in the isolated nanoparticulate assembly, and
(3) subjecting the purified nanoparticle assembly to cryo-EM or NMR to characterize the native structure of the membrane protein.
